(19) United States
(12) Patent Application Publication
(10) Pub. No.: US 2025/0204805 A1
(43) Pub. Date: Jun. 26, 2025

(54) SMART MASK FOR EXHALED BREATH CONDENSATE HARVESTING AND ANALYSIS

(71) Applicant: CALIFORNIA INSTITUTE OF TECHNOLOGY, Pasadena, CA (US)

(72) Inventors: Wei GAO, Pasadena, CA (US); Wenzheng HENG, Pasadena, CA (US)

(21) Appl. No.: 18/999,575

(22) Filed: Dec. 23, 2024

Related U.S. Application Data
(60) Provisional application No. 63/613,186, filed on Dec. 21, 2023.

(51) Int. Cl. A61B 5/097; A61B 5/08; A61M 16/06

(52) U.S. Cl. CPC A61B 5/097; A61B 5/082; A61M 16/0605

(57) ABSTRACT

Systems and methods are provided for a wearable mask that analyzes exhaled breath condensate (EBC) for health monitoring. Specifically, the wearable mask is a smart mask designed to harvest and analyze EBC in real- or near-real-time, and provide insights into the wearer's respiratory and metabolic health. The smart mask incorporates tandem passive cooling technologies, automated microfluidics, selective electrochemical biosensing, and wireless communication within the mask's framework. This integration allows for non-invasive, continuous monitoring of various biomarkers present in the EBC across different environments, enabling personalized health surveillance during regular daily activities. The smart mask is capable of detecting a broad spectrum of biomarkers, including volatile organic compounds (VOCs), nitric oxide, cytokines, and pathogens indicative of respiratory conditions.

### RELATED APPLICATION

This application claims the benefit of U.S. Provisional Application No. 63/613,186 filed on Dec. 21, 2023, the contents of which are incorporated herein by reference in their entirety.

### TECHNICAL FIELD

The present disclosure relates generally to wearable technology for exhaled breath condensate (EBC) analysis. In particular, the disclosed smart mask system integrates tandem passive cooling technologies, automated microfluidics, selective electrochemical biosensing, and wireless communications to enable continuous, multimodal analysis of various EBC biomarkers.

### BACKGROUND

Wearable technologies have experienced a substantial evolution over the past decades, transitioning from simple mechanical devices to sophisticated systems capable of performing a wide range of health monitoring functions. Initially, wearable technologies were largely limited to basic applications, such as tracking physical activity and monitoring heart rate. These early devices laid the groundwork for what has become a burgeoning field of developing technology for personal health monitoring and surveillance. As the potential for wearable devices expanded, so did the ambition to incorporate more complex sensing capabilities, for example, electrochemical sensing or biophysical sensing. These advanced functionalities aim to provide a more comprehensive view of an individual's health by monitoring a variety of biomarkers and vital signs in real-or near-real-time.

The integration of health monitoring functions into everyday items has led to the use of conventional objects, such as face masks, transforming them from simple protective barriers into potential tools for health surveillance. Conventional or traditional face masks have been widely used across various sectors, including healthcare, industry, and general public use, primarily for protection against airborne particles, pathogens, and pollutants. These masks, ranging from simple cloth coverings to more advanced respirators like N95 and surgical masks, serve as physical barriers. However, the evolution of wearable technologies brings forth the possibility of enhancing these traditional face masks with health monitoring capabilities, thereby increasing their utility in both clinical and non-clinical settings.

EBC is a non-invasive, easily obtainable fluid that mirrors the composition of the airway lining fluid. It contains a wide array of biomarkers, including volatile organic compounds (VOCs), inorganic substances, cytokines, and other soluble components. These biomarkers in EBC can provide valuable insights into an individual's respiratory health, metabolic status, and exposure to environmental pollutants. The analysis of EBC biomarkers can offer a promising avenue for early diagnosis, monitoring, and management of various respiratory and systemic diseases.

Traditionally, the measurement of biomarkers in EBC and other bodily fluids involves collecting samples using specialized equipment and then analyzing these samples in a laboratory setting. This process typically requires condenser tubes or specialized condensation instruments to collect EBC, followed by analytical techniques such as mass spectrometry, gas chromatography, or fluorometric assays for biomarker analysis. Although these methods are highly sensitive and specific, they often involve complex procedures, are time-consuming, and require the handling and transport of samples to laboratory facilities for analysis. Moreover, the need for sophisticated equipment and trained personnel further limits the widespread adoption of EBC analysis for routine health monitoring and disease management.

In response to these challenges, there has been a growing interest in developing more accessible, user-friendly technologies that enable real- or near-real-time monitoring of biomarkers directly at the point of care or in the user's natural environment. Such advancements aim to overcome the limitations of traditional EBC collection and analysis methods, offering a more practical approach for continuous health monitoring and early intervention in respiratory and metabolic conditions. This interest aligns with the broader trend of leveraging wearable technologies to enhance personal health surveillance, marking a significant shift towards integrated health monitoring systems embedded within everyday wearables.

### SUMMARY

The convergence of advanced wearable technologies with the analytical abilities of EBC analysis has the potential to significantly improve the field of health surveillance and monitoring. As discussed above, traditional approaches to EBC collection and biomarker analysis, while effective, are often constrained by their complexity, the need for specialized equipment, and their unsuitability for real-time monitoring outside clinical setting. The present disclosure introduces a novel smart mask system, which leverages a combination of tandem passive cooling technologies, automated microfluidics, selective electrochemical biosensing, and wireless communication to facilitate continuous, multimodal monitoring of EBC biomarkers. This innovative system is designed to operate in both indoor and outdoor environments, enabling the real-or near-real-time capture of critical health information during daily activities. By integrating this technology into a wearable device, the present disclosure aims to provide a seamless, user-friendly platform for early diagnosis, health status monitoring, and potential disease management. The disclosed embodiments highlight the feasibility of incorporating sophisticated health monitoring capabilities into everyday wearables and introduce new developments for personalized health surveillance tools accessible to a broader population, thereby enhancing preventative healthcare practices and outcomes.

Embodiments of the present disclosure may include a wearable respiratory analysis system for monitoring and analyzing respiratory emissions. The wearable system may include a mask body, a fluid transport system, a biosensor, a logic circuit, and a transceiver. The mask body may be shaped to cover a portion of a user's face and may be designed to capture respiratory emissions. The mask body may include a cooling layer that condenses the emissions into liquid droplets. The fluid transportation system may connect the mask body to the biosensor, which may include a recognition layer with a bioreceptor and an electrode electrically connected to the layer. The biosensor may generate a signal indicative of the concentration of a biomarker within the liquid droplets and may further transmit this signal to the logic circuit. The logic circuit may then direct the transceiver to send the signal to a remote device, such as a smart phone or medical provider workstation, enabling remote monitoring of the biomarkers detected in the user's respiratory emissions.

Additional embodiments may further discuss the wearable respiratory analysis system. The cooling layer may be passive, and may employ techniques such as evaporative cooling, radiative cooling, thermoelectrical cooling, and/or collecting using a hydrophilic surface material. The fluid transport system may include capillaries with diameters ranging from 1 to 1000 micrometers, and may be designed to facilitate capillary transport of liquid to the biosensor through microchannels. The biosensor may include an electrochemical sensor that can detect various biomarkers, such as nitrites, ammonia, cytokines, acetone, lactate, tuberculosis biomarkers, hydrogen peroxide, nitrates, alcohol, volatile organic compounds, lipids, proteins, DNA, RNA, fatty acids, and viral pathogens. Further, the mask body may incorporate a sunshield made from hybrid metamaterials such as metallic materials, silver, aluminum, oxide materials, titanium dioxide, zirconium dioxide, polymer materials, ceramic polymer-hybrid, aluminum oxide, polyvinylidene fluoride (PVDF), polyethylene (PE), polydimethylsiloxane (PDMS), and/or copolymer PDMS-block-polyethylene glycol (PDMS-b-PEG). The cooling layer may have a structured composition with a hydrogel-based evaporative sub-layer and an aluminum oxide radiative sub-layer within a PDMS matrix. Moreover, the system can include a logic circuit with a processor and memory to execute instructions, enabling the electrode to measure electrical properties of the recognition layer when target biomarkers interact with it.

Embodiments of the present disclosure may include a wearable smart mask analysis system for analyzing EBC for biomarkers. The system may include a mask body of a geometry to cover a user's mouth and nose, with a breath condensation layer for capturing EBC. Integrated within the mask body may be a tandem cooling layer, which may include a hydrogel evaporative cooling sub-layer, a metamaterial radiative cooling sub-layer, and a thermal conductive framework to manage temperature. The system may also feature a microfluidic capillary system designed to capture and transport EBC, utilizing graded capillary channels with a microengineered pillar array and hydrophilic microfluidic channels. For biomarker analysis, a nanoengineered electrochemical biosensor array may be coupled to the microfluidic capillary system, providing selective and sensitive detection. Further, a flexible printed circuit board (FPCB) may interface with the biosensor array, and may facilitate signal processing and enable wireless communication.

Additional embodiments may further discuss the wearable smart mask analysis system. The breath condensation layer may include a hydrophilic surface for effective capture of EBC. The hydrogel evaporative sub-layer may include agarose hydrogel doped with silver nanoparticles, improving its cooling properties. The microfluidic capillary system may further include an evaporative cooling hydrogel top-layer covering the microchannels on the mask body's outer surface to improve temperature regulation. The nanoengineered electrochemical biosensor array may be designed to detect various target biomarkers, such as of nitrites, ammonia, cytokines, acetone, lactate, tuberculosis biomarkers, hydrogen peroxide, nitrates, alcohol, volatile organic compounds, lipids, proteins, DNA, RNA, fatty acids, and viral pathogens. Further, the system may include a recognition layer with a bioreceptor, such as an enzyme or antibody, that selectively interacts with target biomarkers. An electrode may be configured to measure electrical properties of the recognition layer, and a logic circuit with a processor and memory may be included to process these measurements. Moreover, the system may include a sunshield layer made from a metamaterial, such as a ceramic alumina-polymer hybrid or similar material, providing for additional protection and functionality.

Embodiments of the present disclosure may include a method for analyzing EBC using a wearable mask of a geometry to cover a user's respiratory outlets. The mask may include a passive cooling system for condensing exhaled breath into EBC, a microfluidic system for directing EBC to a designated analysis area, an integrated biosensor array for detecting biomarkers, and a communication module for transmitting analysis results. The method may include capturing and condensing the exhaled breath into EBC using the passive cooling system, which may include a tandem cooling layer with an evaporative hydrogel sub-layer, a radiative cooling sub-layer, and a hydrophilic surface material. The condensed EBC may then be transported via the microfluidic system to the biosensor array using fluid capillary forces generated by graded pillar structures. In situ analysis of the EBC may be performed by the biosensor array to detect and quantify the biomarkers indicative of the user's respiratory health. The analysis results may then be transmitted to an external receiver through the communication module. Further, the hydrogel sub-layer of the passive cooling system may be refreshed through the microfluidic transport of EBC, maintaining its effectiveness.

Additional embodiments may further discuss the method for EBC analysis. The passive cooling system's evaporative cooling hydrogel sub-layer may be improved by introducing a microbial agent, increasing its hygiene and safety. The analysis of EBC may involve using electrochemical sensors within the biosensor array to perform multiplexed analysis, allowing for the detection of multiple biomarkers. Prior to analysis, the biosensor array may be calibrated by adjusting sensor responses based on known concentrations of analytes, improving the accuracy of measurements. The method of transporting condensed EBC through the microfluidic system to the integrated sensor array may incorporate a microengineered gradient in pillar height and density, which may facilitate efficient fluid movement. Moreover, the results of the EBC analysis may be transmitted to an external device using a low-energy wireless protocol, optimizing power consumption while maintaining effective data communication.

Other features and aspects of the disclosure will become apparent from the following detailed description, taken in conjunction with the accompanying drawings, which illustrate, by way of example, the features in accordance with various embodiments. The summary is not intended to limit the scope of the invention, which is defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is an illustration showing two expanded views of an example wearable respiratory analysis system without a mask body, in accordance with various embodiments of the disclosed technology.

FIG. 2 is an illustration showing a third expanded view of an example wearable respiratory analysis system, in accordance with various embodiments of the disclosed technology.

FIG. 3 is an illustration showing a perspective and plan view of an example wearable respiratory analysis system, in accordance with various embodiments of the disclosed technology.

FIG. 4 is an illustration showing a second plan view of an example wearable respiratory analysis system including various expanded features of the system, in accordance with various embodiments of the disclosed technology.

FIG. 5 is an illustration showing a perspective view of an example electrochemical biosensor array, in accordance with various embodiments of the disclosed technology.

FIG. 6A is an illustration showing a second perspective view of an example wearable respiratory analysis system, in accordance with various embodiments of the disclosed technology.

FIG. 6B is an illustration showing a front plan view of an example wearable respiratory analysis system, in accordance with various embodiments of the disclosed technology.

FIG. 6C is an illustration showing a perspective view of exemplary inner surface graded micropillars, in accordance with various embodiments of the disclosed technology.

FIG. 6D is an illustration showing a perspective view of exemplary outer surface microchannels, in accordance with various embodiments of the disclosed technology.

FIG. 6E is an illustration showing front and rear perspective views of an example biosensing reservoir, in accordance with various embodiments of the disclosed technology.

FIG. 7 is an illustration showing a perspective view of an example wearable respiratory analysis system worn by a user, in accordance with various embodiments of the disclosed technology.

FIG. 8A is an illustration showing an inner plan view of exemplary density gradient parameters of micropillars in the wearable respiratory analysis system, in accordance with various embodiments of the disclosed technology.

FIG. 8B is an illustration showing a side plan view of exemplary height gradient parameters of micropillars in the wearable respiratory analysis system, in accordance with various embodiments of the disclosed technology.

FIG. 8C is an illustration showing an outer plan view of example microchannels of the wearable respiratory analysis system, in accordance with various embodiments of the disclosed technology.

FIG. 9 is an illustration showing an example fabrication method for the wearable respiratory analysis system, in accordance with various embodiments of the disclosed technology.

FIG. 10 is an illustration showing an example flexible printed circuit board (FPCB), in accordance with various embodiments of the disclosed technology.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

Recent respiratory outbreaks have highlighted the need for comprehensive monitoring, yet most current methods focus only on physical signals. Exhaled breath condensate (EBC) offers a wealth of molecular information that can provide valuable insights into an individual's health. However, challenges with sample collection and the absence of on-site analytical tools have hindered the widespread use of EBC analysis. This disclosure presents embodiments of a wearable respiratory analysis system, a mask-based device for real-time or near-real-time in situ monitoring of EBC biomarkers. Various embodiments of the disclosure may feature a single or tandem cooling strategy, automated microfluidics, selective electrochemical biosensors, and wireless circuits, enabling continuous multimodal monitoring of EBC analytes during everyday activities, both indoors and outdoors. The system may be particularly beneficial for patients with a range of metabolic and respiratory conditions, such as airway inflammation, chronic obstructive pulmonary disease (COPD), asthma, and those with or recovering from COVID-19.

Respiratory epidemics and pandemics have emphasized the urgent and critical need for comprehensive research on the respiratory system. A variety of clinically meaningful molecular analytes, such as volatile organic compounds (VOC, e.g., acetone and alkanes), inorganic substances (e.g., nitric oxide and ammonia), cytokines, and pathogens (e.g., severe acute respiratory syndrome coronavirus) may be exhaled in the form of gases, aerosols, or droplets. These analytes may also be referred to as biomarkers throughout this disclosure. The real-time and continuous analysis of exhaled breath biomarkers can provide potential for offering valuable insights into the early diagnosis, monitoring, and management of a variety of respiratory and metabolic health conditions, including asthma, chronic obstructive pulmonary disease (COPD), lung cancer, tuberculosis, and other pulmonary conditions. Despite this promise, conventional tools for studying human breath remain very limited. Existing methods of real-time and wireless monitoring of exhaled breath molecular biomarkers are often limited to breath alcohol tests, and “electronic noses” based on gas sensors. Although these conventional methods are actively researched, they are limited by inadequate selectivity for practical exhaled breath analysis.

EBC is a promising noninvasive aqueous matrix that soluble gaseous and nonvolatile biomarkers can be measured selectively from for personalized healthcare. Clinically, EBC may be collected using a commercial condenser or specialized condensation instruments and subsequently analyzed in laboratory settings by means of mass spectrometry of photometric assays to assess airway inflammation and substance metabolism. The implementation of these approaches for at-home remote sensing is hindered by challenges related to labor, time, money, and energy. Furthermore, issues such as the degradation of reactive substance (e.g., nitrite (NO₂⁻) and hydrogen peroxide (H₂O₂)) during the sampling process and storage, interference of oral ingredients, and the absence of continuous dynamic information impede the practical and widespread application of EBC testing using conventional methods.

Recent developments of wearable biosensors have ushered in a new era of telehealth, enabling continuous and wireless molecular monitoring of biomarkers in sweat, saliva, and interstitial fluid. The exploration of wearable EBC analysis has been limited, however, primarily because of the challenges in condensing exhaled breath, EBC sampling, and in situ analysis during an individual's daily activities.

Face masks are an ideal wearable platform for personal protection and breath sampling. Recent advances in exhaled breath aerosol (EBA) devices based on masks have shown some promise in point-of-care analysis, but their reliance on external media for sample extraction has introduced challenges in terms of stability and reproducibility, which limits their suitability for continuous monitoring.

The present disclosure responds to these challenges and introduces a mechanically soft microfluidic smart mask system, which can conduct EBC analysis and respiratory evaluation (or EBCare), designed for continuous exhaled breath condensation, automatic EBC capturing and transport, and real- or near-real-time in situ biomarkers analysis. Throughout this disclosure, the smart mask system may be referred to as a wearable respiratory analysis system, a wearable smart mask analysis system, and/or a smart mask. The smart mask may include two distinct parts, the first being the mask body and the second being an EBCare device secured through a mounting hole on the mask body.

Compared to bulky traditional EBC collection devices that rely heavily on ice buckets or refrigeration, the present wearable respiratory analysis system may be capable of effective condensation of breath vapor in both indoor and outdoor environments through single or tandem passive cooling technologies that may integrate hydrogel evaporative cooling, metamaterial radiative cooling, and/or a device framework with a high thermal conductivity.

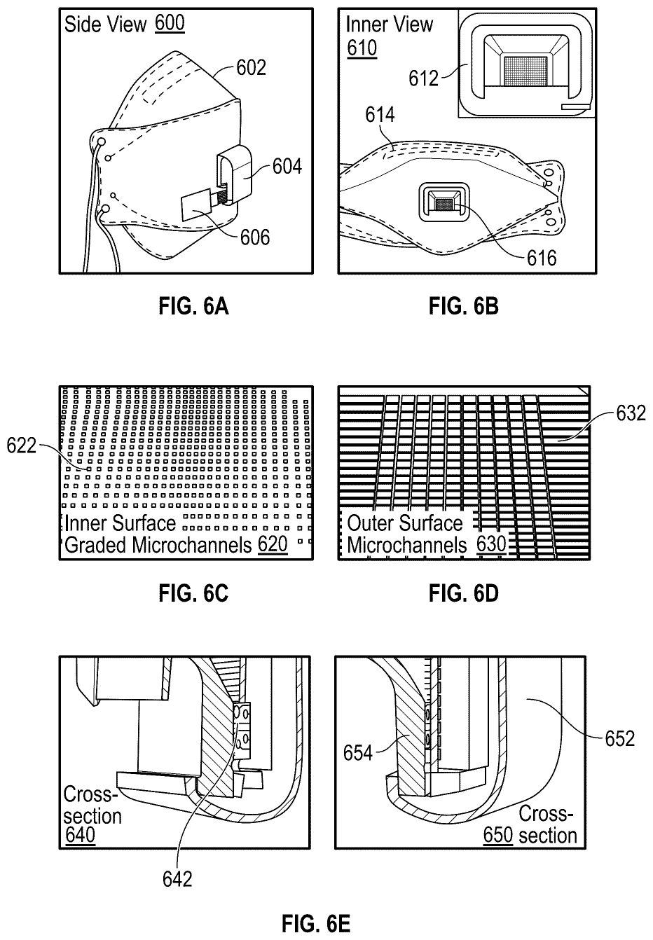

Further, a bioinspired microfluidic module may improve EBC harvesting and transport efficiency by leveraging the capillary action driver by surface hydrophilicity and a micro-engineered graded pillar array (shown and discussed in relation to FIG. 6C below). The disclosed wearable respiratory analysis system may support high-temporal-resolution EBC harvesting and transport, making it viable for real-time continuous in situ analysis. The present system may enable sensitive, selective, and continuous EBC biomarker analysis, facilitated by a nanoengineered electrochemical biosensor array coupled with a flexible printed circuit board (FPCB) for signal processing and wireless communication. The post-analysis EBC efflux may be absorbed by a cooling hydrogel, ensuring a continuous water replenishment for sustainable evaporative cooling.

EBC harvesting plays a foundational role in achieving real-time and continuous EBC biomarker analysis with high temporal resolution. To efficiently cool down the condensing surface temperature to the dew point of the exhaled breath in diverse real-life indoor and outdoor scenarios, the wearable respiratory analysis system may use a tandem passive cooling strategy, combining hydrogel evaporation and radiative cooling. The structural framework of the wearable respiratory analysis system may use a ceramic alumina-polymer hybrid metamaterial with a high thermal conductivity and high radiative cooling properties, comprising micrometer-sized aluminum oxide (Al₂O₃) spheres distributed approximately evenly in a polymeric matrix including polydimethylsiloxane (PDMS) and copolymer PDMS-block-polyethylene glycol (PDMS-b-PEG). These compounds are exemplary, other compounds or materials may be used to construct the wearable respiratory analysis system.

During operation in ambient conditions, the natural evaporation of water from an agarose hydrogel may absorb surrounding heat, reducing the temperature of the hydrogel. The addition of silver (Ag) nanoparticles into the hydrogel may both introduce a high antibiotic effect and an improved biocompatibility during long-term on-body use. In other words, the addition or infusion of the silver nanoparticles may function as the addition of an antimicrobial agent to the hydrogel.

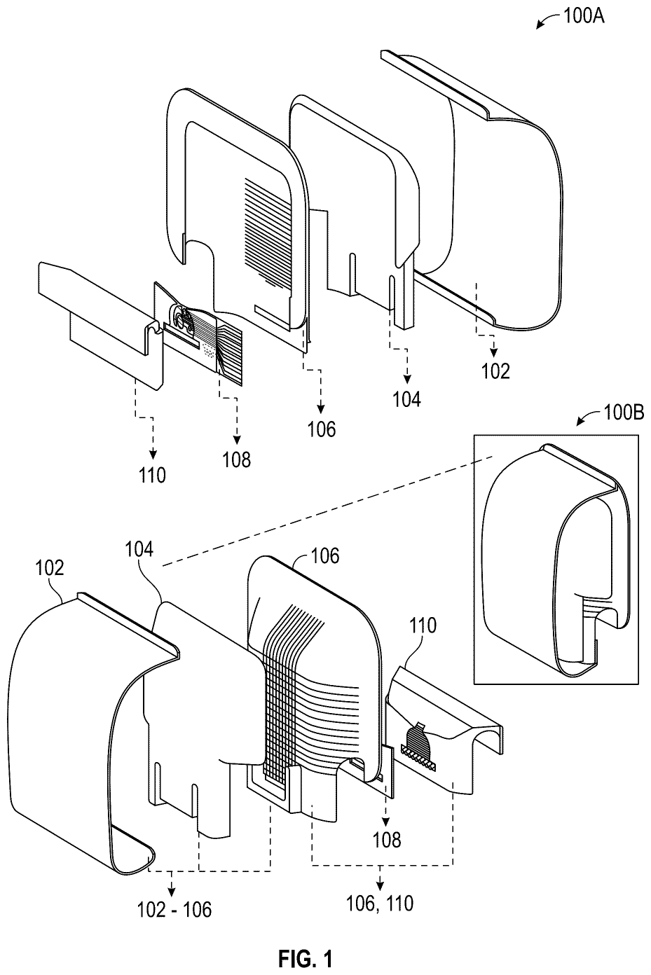

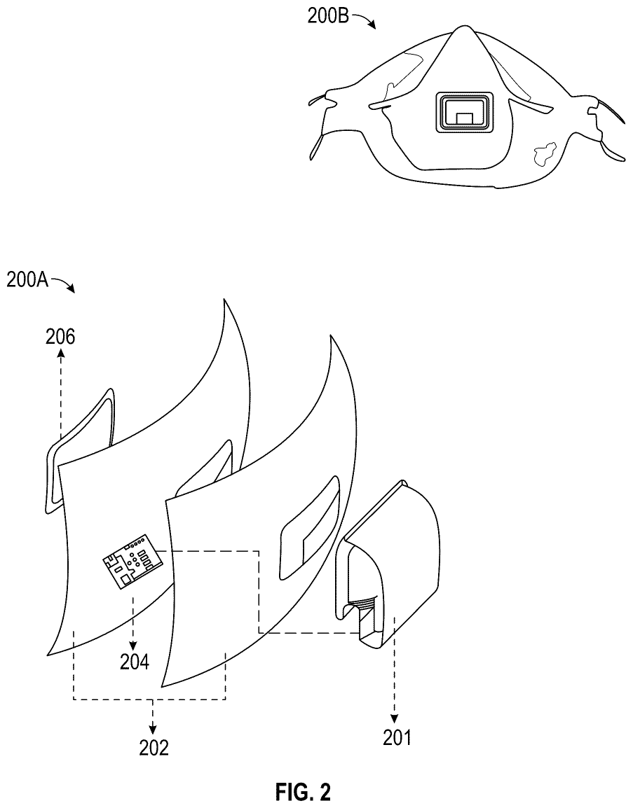

Turning now to the figures. As discussed, the wearable respiratory analysis system may be separated into two features. First, as shown in FIG. 2, may be the mask layers 202, which form a mask body, and second, may be the EBCare device 100A, 100B, which may be the combination of the cooling layers 102-106, the microfluidics layers 106, 110, and the sensing layer 108, as shown in FIG. 1. Additionally, a FPCB may be secured between mask layers and electrically connected to the EBCare device, as such, the FPCB may be discussed in combination with either feature or alone, as a third feature.

Accordingly, FIG. 1 is an illustration showing two expanded views of an example wearable respiratory analysis system without a mask body (also referred to as the EBCare device 100A, 100B), in accordance with various embodiments of the disclosed technology. The EBCare device is one of the two discussed features of the wearable respiratory system, designed for efficient capture and analysis of EBC. EBCare device 100A provides an illustration of the front view or interior of the EBCare device 100A, depicting internal features that facilitate the collection and initial processing of EBC. For example, this view depicts the placement of micropillars in the microfluidic layer 106 for directing the EBC towards the biosensors 108. EBCare device 100B, on the other hand, provides an illustration of the back view or exterior of the EBCare device 100B. This perspective shows the structural components and external features that support the mask's functionality and improve user comfort. The exterior design may include elements such as microchannels on the microfluidic layer 106, sun protection on the sunshield layer 102, and/or external interfaces for data transmission or power supply.

The EBCare device 100A, 100B may integrate into the mask body, allowing for real-time monitoring of respiratory biomarkers, when the combined EBCare device/mask body is worn by a user. Its interior components, as depicted by EBCare device 100A, may capture moisture-laden breath, cool the breath using the cooling layers 102-106 such that the breath is condensed into droplets, which are aggregated as EBC and channeled through the fluid transport system (e.g., micropillars and capillary veins) 106, 110 to a sensing layer containing at least one biosensor 108. The EBCare device's 100B exterior may include a durable outer shell (or sunshield layer 102) that provides protection from physical damage as well as sun damage for the sensitive internal components while maintaining a lightweight and comfortable fit for the user.

The EBCare device 100A, 100B may include a series of cooling layers 102-106, each designed to perform a cooling function for effective capture and analysis of EBC. The first layer 102, comprised of PDMS: PDMS-b-PEG/Al₃O₃, may provide for radiative cooling, helping to dissipate heat through emission of thermal radiation. This layer may also be referred to as the sunshield layer 102 as it protects the EBCare device 100A, 100B from solar emissions. The next layer, an Agarose/Ag nanoparticles layer 104, may serve as an evaporative cooling layer, which functions by absorbing heat as the liquid in hydrogel evaporates, enhancing the cooling effect and aiding in the aggregation of EBC. The second PDMS: PDMS-b-PEG/Al₃O₃ layer 106, similar to the first, contributes to radiative cooling. However, this layer is also equipped with micropillars, which are used for capillary transport of EBC droplets through fluidic channels, improving efficient movement of EBC towards the biosensor 108. Alternative embodiments of the EBCare device 100A, 100B may include a different number of layers than those described, or various combinations of layers tailored to achieve distinct cooling effects. These configurations can incorporate different materials within the component layers to produce a wide range of thermal management solutions, adapting the device to diverse environmental conditions and user needs.

The EBCare device 100A, 100B may further include condensation and microfluidic layers, specifically layers 106 and 110, for effective management and transport of EBC. These layers include the second PDMS: PDMS-b-PEG/Al₃O₃ layer 106 and a PDMS: PDMS-b-PEG layer 110. These materials may be chosen for their thermal and hydrophilic properties, which facilitate efficient condensation and fluid transport. The PDMS: PDMS-b-PEG/Al₃O₃ layer 106 may include micropillars on its inner surface. These micropillars may enhance the condensation process by increasing the surface area available for collecting moisture from the exhaled breath. They also play a role in directing the condensed droplets into the fluid transport system or towards the biosensor 108. On the outer surface, microchannels may be engineered to optimize the movement of EBC through the device via efflux transmission, further contributing to the evaporative cooling effect. These channels are graded, meaning they have varying widths and depths, which helps maintain a capillary action that continuously draws the condensate towards the biosensor array.

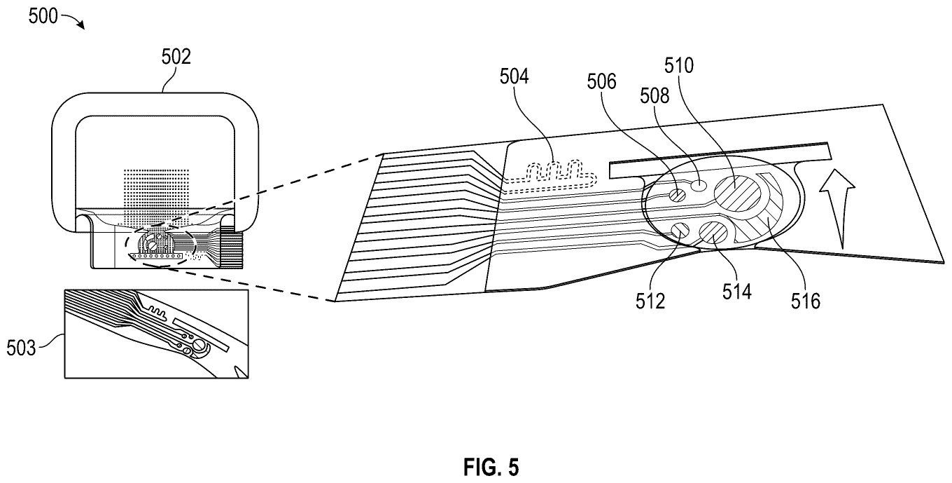

Layer 110, made from PDMS: PDMS-b-PEG, complements this process by further refining the transport of EBC through its hydrophilic properties, which help maintain a smooth and consistent flow of condensate across the surface. Embedded within these layers is the sensing layer 108, which houses the electrochemical biosensor array. This array, exemplified by the electrical biosensor array 503 shown in FIG. 5, is responsible for analyzing the EBC for various biomarkers. This setup ensures that as EBC is efficiently collected and transported, it is quickly analyzed in situ, providing real-time data about the user's respiratory health.

Additional embodiments of the EBCare device 100A, 100B may feature a different number of layers or alternative combinations of materials, tailored to achieve specific effects in condensation and fluid transport. These variations allow the device to be adaptable to different environmental conditions and user needs, ensuring optimal performance across a range of scenarios.

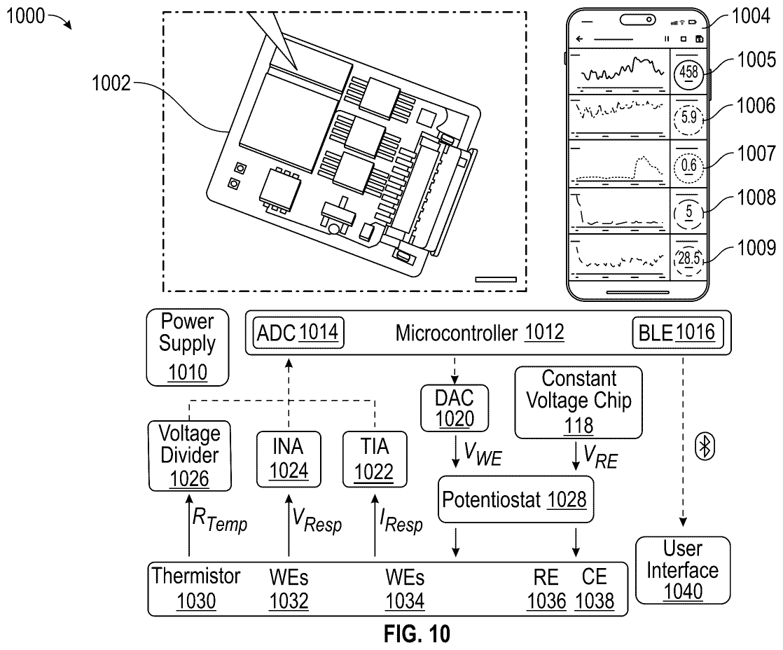

FIG. 2 is an illustration showing a third expanded view of an example wearable respiratory analysis system 200A and a non-expanded, interior view of an example wearable respiratory system 200B, in accordance with various embodiments of the disclosed technology. The wearable respiratory analysis system 200A may include an EBCare device 201 (e.g., the EBCare device 100A, 100B discussed with relation to FIG. 1), mask body layers 202, a flexible printed circuit board 204 (FPCB, discussed in relation to FIG. 10), and medical tape 206.

In particular, the mask body layers 202 may include a first and second layer. A FPCB 204 may be secured between the first and second mask body layers 202, such that the FPCB is hidden from view when the mask is worn by a user. Both layer of the mask body layers 202 may have mounting holes cut through them to place the EBCare device 201 therein. The mounting holes may be cut in corresponding heights and widths to the geometry of a perimeter of the EBCare device 201, such that when the EBCare device 201 is placed in the mounting hole, the walls of the mounting hole physically contact the perimeter of the EBCare device. Furthermore, the medical tape 206 may secure the EBCare device 201 to the mask body 202.

The wearable respiratory analysis system 200B may illustrate the expanded layers of wearable respiratory analysis system 200A, in a non-expanded form.

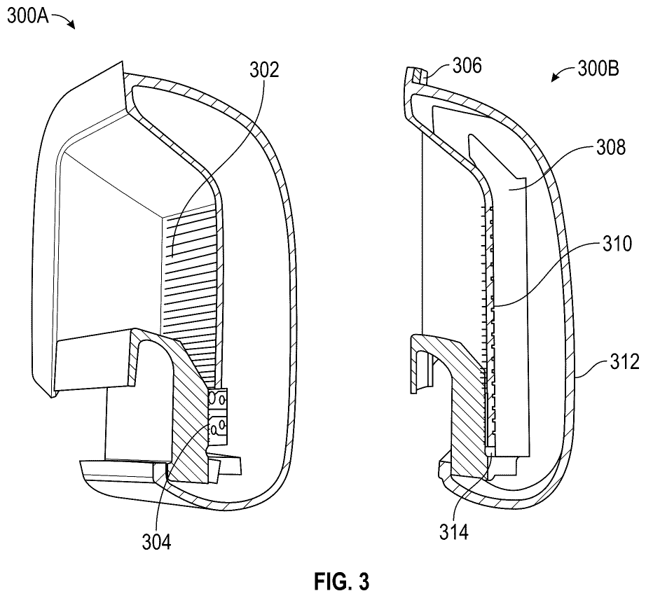

FIG. 3 is an illustration showing a perspective 300A and plan view 300B of an example wearable respiratory analysis system, in accordance with various embodiments of the disclosed technology. Embodiments of the wearable respiratory analysis system 300A may include graded micropillars 302 and a sensing reservoir 304. Further embodiments of the wearable respiratory analysis system 300B may also include a sealing edge 306 that may seal the EBCare device around its perimeter to the mounting hole of the mask body, cooling hydrogel 308 that can contribute to the evaporative cooling effect, microchannels 310 for microfluidic capillary EBC transport, a sunshield layer 312, and efflux pillars 314.

On sunny days, under strong solar radiation, the potential elevation of the wearable respiratory analysis system's temperature as a result of the hydrogel absorbing sunlight could compromise breath condensation during outdoor use. To address this issue, a tandem radiative cooling function may be integrated into the system using a metamaterial, such as PDMS: PDMS-b-PEG/Al₂O₃, as the main framework and/or as a sunshield layer 312. Owing to the Mie scattering of spherical alumina microparticles under solar spectrum and the strong emission properties of polymeric matrix in the mid-infrared (MIR) region, the hybrid polymeric metamaterial may achieve 85-95% solar reflectivity and 85-95% MIR thermal emissivity. Such optical properties may reduce the solar radiation absorption and radiate heat through the MIR range in the atmospheric window to outer space, improving the efficiency of the condensation process even under strong sunlight exposure in outdoor environments.

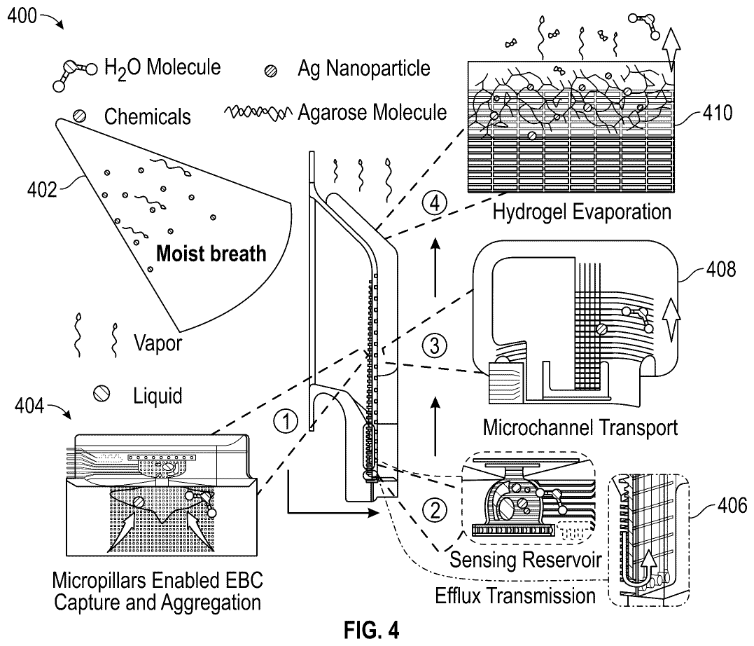

FIG. 4 is an illustration showing a second plan view of an example wearable respiratory analysis system 400 including various expanded features of the system and the methods of EBC transport associated with said features, in accordance with various embodiments of the disclosed technology. The wearable respiratory analysis system 400 may capture moist breath 402 exhaled from a user's mouth or nose. This exhaled breath may contain a variety of analytes or biomarkers, which may be measured by the electrochemical sensor array or sensing layer to assess the user's respiratory and metabolic health. Within the EBCare device, an array of micropillars may be used to capture and aggregate the EBC 404. The micropillars and microchannels discussed herein may makeup the wearable respiratory analysis system's fluid transport system. These micropillars may be engineered to maximize the collection of EBC, ensuring that a sufficient volume is gathered for accurate analysis. Once captured, the EBC may be transported to a sensing reservoir (e.g., sensing reservoir 304 of FIG. 3), wherein it may undergo a biomarker analysis to determine concentration and/or presence of target biomarkers. From the sensing reservoir, post- analysis, the EBC may undergo efflux transmission 406 via the efflux pillars. Following this, the EBC may be directed through a network of microchannels 408, which may facilitate capillary transport. As the EBC travels through these channels, it may encounter a hydrogel. The combination of EBC with the hydrogel may lead to hydrogel evaporation 410.

The continuous respiratory monitoring capability of the wearable respiratory analysis system may be based on the self-directed flow of EBC within an integrated bioinspired microfluidic system (which may include the various features described with relation to 404-410). The microfluidic system may function similarly to the natural conveyance of water and chemicals in plants that may rely on the capillary phenomenon. In plants, water may transpire from the stomata of leaves, transforming into water vapor. This process induces a reduction of water content inside the leaves, creating negative pressure inside the plant's diminutive hydrophilic xylem vessels. Consequently, the water is drawn upward from the ground through the capillary forces to meet the plant's water needs. Similarly, the wearable respiratory analysis system's microfluidic or fluid transport system may incorporate micropillars with structural gradients, hydrophilic microfluidic channels, and evaporative cooling hydrogels, serving as the graded capillary pumps for gravity-independent EBC sampling, transport, and refreshing.

The hydrophilic interface of the wearable respiratory analysis system's inner surface, which may be similar to xylem vessels, may enable the automatic circulation of harvested EBC through microfluidic channels. The hydrophilic surface may be a polydimethylsiloxane (PDMS) and copolymer PDMS-block-polyethylene glycol (PDMS-b-PEG) and/or plasma treated PDMS and/or silicone. In some embodiments, increasing the doping ratio of the copolymer PDMS-b-PEG to 1% in the wearable respiratory analysis system's framework material (PDMS: PDMS-b-PEG/Al₂O₃) improved hydrophilicity, and further may achieve a low contact angle of 12-18° owing to an increased carbon-oxygen bond surface distribution. Unlike plasma-treated pristine PDMS, which can lose its hydrophilicity within hours, the hybrid polymer PDMS: PDMS-b-PEG/Al₂O₃ may maintain high hydrophilicity over a longer period, for example, up to one month. This hydrophilic nature, in contrast to hydrophobic surfaces, can offer advantages in terms of EBC nucleation, cohesion, and collection. Such a hybrid material also may exhibit biological anti-adhesive and non-fouling properties, making it suitable for EBC sampling and subsequent in situ bioanalysis with improved accuracy.

The transport of EBC to the sensing reservoir, after EBC capture at the hydrophilic inner surface of the wearable respiratory analysis system, may be facilitated by the graded capillary forces resulting from an array of micropillars with both height and density gradients. The stabilized EBC may continuously flow through the sensing reservoir, where it can be analyzed by electrochemical sensors. Subsequently, driven by strong capillary forces from hydrogel-covered microchannels on the wearable respiratory analysis system's outer surface, EBC may automatically transfer to the device's outer surface through efflux columns between the inner and outer interfaces and transport against gravity via microfluidic channels. EBC may then be absorbed by the hydrogel along with the hydrogel evaporation, providing a continuous water source for hydrogel evaporative cooling.

Experimentation revealed that the wearable respiratory analysis system made of pristine PDMS was not capable of sampling EBC into the sensing reservoir under both upright and supine conditions owing to the hydrophobic surface. The wearable respiratory analysis system with planar hydrophilic PDMS: PDMS-b-PEG/Al₂O₃ was only able to access EBC under upright conditions, where gravity served as the driving force. In contrast, the wearable respiratory analysis system, constructed from hydrophilic PDMS: PDMS-b-PEG/Al₂O₃ with graded microstructures, demonstrated the ability to harvest and transport EBC into the sensing reservoir within a discrete time period even in supine postures.

Stable and continuous wearable EBC analysis may be possible under various real-life conditions, including gravity-defying supine positions, where graded capillary forces act as the primary driving force. On the basis of this design, the wearable respiratory analysis system's start-up time for in situ EBC analysis may be approximately 1-5 minutes when in an upright posture. Further, the wearable respiratory analysis system can harvest EBC from human participants at rates as high as approximately 5 μl min⁻¹. This rate may be sufficient for high-temporal-resolution EBC analysis, given the small volume of the sensing reservoir (approximately 5 μl).

FIG. 5 is an illustration showing a perspective view of an example electrochemical biosensor array 503, in accordance with various embodiments of the disclosed technology. This biosensor array 503 may be part of the wearable respiratory analysis system 500, integrated into the EBCare device 502 for simultaneous and multiplexed in situ analysis of EBC. The electrochemical biosensor array 503 may be mechanically flexible and disposable, featuring various sensors, including: a resistive temperature sensor 504, a potentiometric ion-selective pH sensor 506, an amperometric nitrite (NO₂) sensor 510, an NH₄⁺ sensor 512, and an alcohol sensor 514. Additionally, the system may incorporate other sensors for detecting a wide range of biomarkers, such as nitrites, ammonia, cytokines, acetone, lactate, tuberculosis biomarkers, hydrogen peroxide, nitrates, alcohol, volatile organic compounds, lipids, proteins, DNA, RNA, fatty acids, viral pathogens, and other clinically significant biomarkers.

The electrochemical biosensor array 503 may include a plurality of biosensors, wherein each biosensor may be configured to detect a different analyte or biomarker. Each biosensor may have a recognition layer, which may be the top layer of the sensor that directly interacts with and identifies the target analyte. Each recognition layer may include a bioreceptor, which may include an enzyme, an ion-selective molecule, an imprinted polymer, an antibody, metallic materials (e.g., silver and/or aluminum), and/or similar, and an electrode electrically coupled to the recognition layer. Each biosensor may include a working electrode, which can be compared to a reference electrode.

The electrochemical biosensor array 503 may also include a reference electrode (RE) 508 and a control electrode (CE) 516. The RE 508 may provide a stable potential for improved accuracy in measurements, while the CE 516 may complete the circuit by balancing the current flow, both being important for reliable electrochemical analysis in the sensor array 508.

The electrochemical biosensor array 503 can be cost-effectively mass-produced using inkjet printing on a flexible substrate like polyethylene terephthalate (PET).

EBC may be characterized as a highly diluted solution with low ionic strength, where NH₄⁺ is the dominant cation, influencing its electrical conductivity. The electrochemical biosensor array 503 may be designed to be highly effective for analyzing EBC. This may involve optimizing the array to ensure it has a linear range suitable for detecting physiological concentrations, while also maintaining stability in environments with low ionic strength. The selection of target analytes and biomarkers may be based on their clinical significance. For example, alcohol detection may be achieved using a platinum-decorated gold electrode modified with alcohol oxidase. Nitrite levels may be quantified through selective oxidation at an applied redox potential of about 0.75 V on an inkjet-printed carbon nanoparticle electrode. Additionally, pH and NH₄⁺ levels may be monitored using a pH-responsive polyaniline film and NH₄⁺ selective membrane-modified electrodes, respectively.

The electrochemical characterization of each biosensor in the electrochemical biosensor array 503 may involve testing in standard solutions containing analytes at physiologically relevant concentrations. This may be demonstrated by linear responses in current signals for alcohol and NO₂⁻ and voltage signals for pH and NH₄⁺ sensors, which correlate with the target concentrations. The biosensors may exhibit selectivity in EBC, minimizing interference from other analytes. While pH and NH₄⁺ levels may provide clinically relevant data, they also help calibrate the alcohol and nitrite sensors. Furthermore, real-time temperature data from the integrated carbon-based resistive temperature sensor 504 may enhance sensor calibration during device use. These calibration mechanisms ensure that the NH₄⁺ 512 and NO₂⁼ sensors 510 are highly accurate for analyzing human EBC, with their performance validated and further calibrated against known analyte concentrations.

FIG. 6A is an illustration showing a second perspective view of an example wearable respiratory analysis system 600, in accordance with various embodiments of the disclosed technology. Similarly, the wearable respiratory analysis system 600 may include a mask body 602 with an EBCare device 602 disposed therein, which is further electronically attached to a FPCB 606.

FIG. 6B is an illustration showing a front plan view of an example wearable respiratory analysis system 610, in accordance with various embodiments of the disclosed technology. Similarly, the wearable respiratory analysis system 610 may include a mask body 614 with an EBCare device 616 disposed therein. Some embodiments may include an open EBCare structure 612, such that when the mask is worn, EBC is collected into the open EBCare device structure.

FIG. 6C is an illustration showing a perspective view 620 of exemplary inner surface graded micropillars 622, in accordance with various embodiments of the disclosed technology. FIG. 6D is an illustration showing a perspective view 630 of exemplary outer surface microchannels 632, in accordance with various embodiments of the disclosed technology. FIG. 6E is an illustration showing front 640 and rear 650 cross-section perspective views of an example biosensing reservoir 642, 654, in accordance with various embodiments of the disclosed technology. Cross-section 650 also depicts the sunshield layer 652 as discussed in relation to FIGS. 1 and 3.

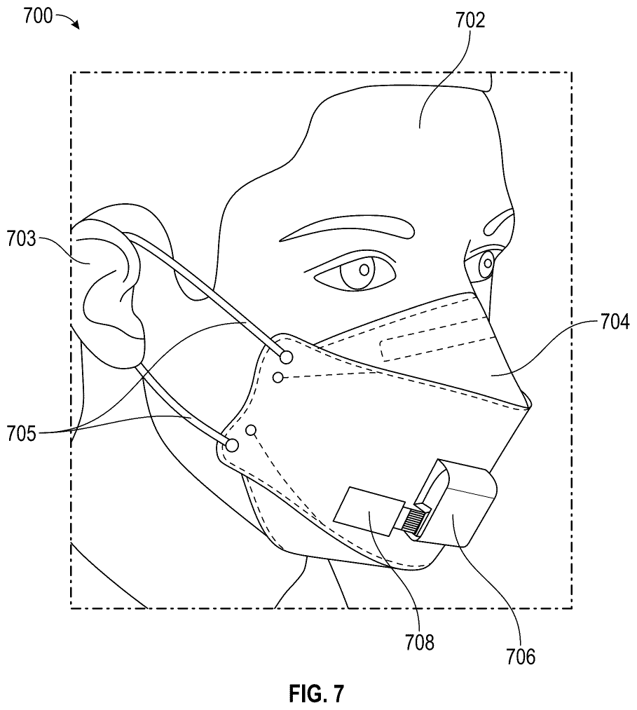

FIG. 7 is an illustration showing a perspective view of an example wearable respiratory analysis system 700 worn by a user, in accordance with various embodiments of the disclosed technology. The wearable respiratory analysis system 700 may include a mask body 704, which may be of a geometry or shape to cover specific portions of the user's face. In various embodiments, the mask body 704 may be shaped to simultaneously cover both the user's mouth and nose, providing coverage for capturing respiratory emissions. Alternatively, the mask may be configured to cover only the mouth, leaving the nose exposed, which could be beneficial in scenarios where nasal breathing needs to remain unimpeded or uncaptured. This flexibility in design allows the mask to be adapted for various user preferences and needs.

To ensure a secure fit, the mask body 704 may be equipped with straps 705. These straps 705 may be designed to loop around the user's ears 703, providing a comfortable and stable attachment that keeps the mask attached to the user's face 702 during use. This design is particularly advantageous for disposable mask variants, which prioritize ease of use and comfort for short-term applications.

In addition to these embodiments, alternative embodiments may incorporate features such as adjustable straps for a customizable fit, or enhanced sealing materials around the edges of the mask to improve capture efficiency of EBC. Some embodiments may also include integrated nose clips for better fit and reduced leakage, or replaceable filter inserts for prolonged use in specific environments. These variations highlight the adaptability of the mask system to meet diverse user requirements and environmental conditions.

The wearable respiratory analysis system 700 may also include an EBCare device 706, for example the EBCare device 100A, 100B described in relation to FIG. 1. The wearable respiratory analysis system 700 may further include a flexible printed circuit board 708 (FPCB), which may be interfaced with the electrochemical biosensor array of the EBCare device 706 for signal processing and wireless communication.

As discussed, the wearable respiratory analysis system 700 may utilize various types of face masks 704 for efficient EBC sampling due to their high mechanical flexibility and stretchability. It should be noted that placing the EBCare device 706 above the upper lip when integrated with a mask body 704 may mitigate saliva contamination in EBC sampling, as can be shown by saliva amylase tests on collected EBCs. In cases of transient or long-term coughing, saliva contamination effects on EBC analyte concentration can be eliminated within a few minutes through microfluidic refreshing, as can be shown in simulations and on-body test results.

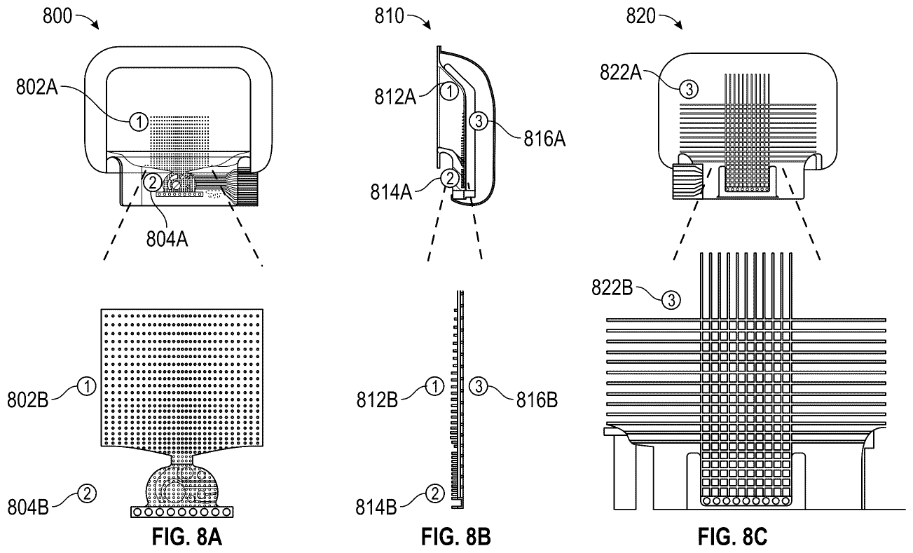

FIG. 8A-8C are illustrations showing an inner plan, side plan, and outer plan view, respectively, of exemplary density gradient parameters of micropillars and microchannels in the wearable respiratory analysis system, in accordance with various embodiments of the disclosed technology. In particular, the wearable respiratory analysis system 800 depicts density gradient parameters of micropillars, whereas the wearable respiratory analysis system 810 depicts height gradient parameters of micropillars along the median axis of the inner surface of an EBCare device. Further, the wearable respiratory analysis system 820 depicts the parameters of the microchannels of the EBCare outer surface.

The design of the EBCare microstructure can be divided into three parts (inner, reservoir, and outer structures). The overall design principle may recognize that the capillary pressure should be: reservoir>outer surface>inner surface. The inner surface may have a graded micropillars array 802A, 802B for gravity-independent self-aggerating of EBC liquid to the biosensing reservoir 804A, 804B. In the first part, the microstructure of EBCare's inner surface may be a micropillar array with a height and density composite gradient. The diameter of micropillars may be the minimum feature size of the 3D printer, e.g., 200 μm. The graded density distribution of the micropillars may be achieved using a two-dimensional (2D) circular array.

The density may be the highest at the origin point in FIG. 8A (e.g., a 200 μm spacing of the micropillars and a density of 625 pillars cm⁻²). Subsequently, the spacing of the pillars may be increased by, for example, 20 μm per pillar along the x- and y-axis until reaching the edge of the internal interface, thus the density of the micropillars may decrease progressively. After importing the 2D array, it may be extruded to form a 3D micropillar array with a height. To create the height gradient, three planes (A, A′, and B) may be generated by positioning along the z-axis on the sides (left, right, and top) and at the origin. The 3D pillars above these planes may then be deleted. Through these operations, an array of EBCare micropillars with both height and density gradients may be formed on the inner surface.

The second part of the EBCare device 810 may be the micropillar 812A, 812B and efflux structure 816A, 816B inside the biosensing reservoir 814A, 814B. The pillars' density inside may be the highest (e.g., 625 pillars cm⁻²). The distance from the bottom biosensor to the top of the pillars may be, for example, 200 μm. The efflux pillars 816A, 816B for internal and external flow connection may have the same spacing of, for example, 200 μm from the pillars inside the chamber. The height of these efflux pillars 816A, 816B may be designed to contact the hydrogel.

The third section of the EBCare device 820 may include external microchannels 822A, 822B with a spacing of, for example, 800 μm. The cross-sections of these channels may be open squares with sides measuring, for example, 200 μm, and pieces of cooling hydrogel may be attached at the top of these channels. Lengths disclosed within this disclosure are merely exemplary and for purposes of discussion, other lengths of component features may exist.

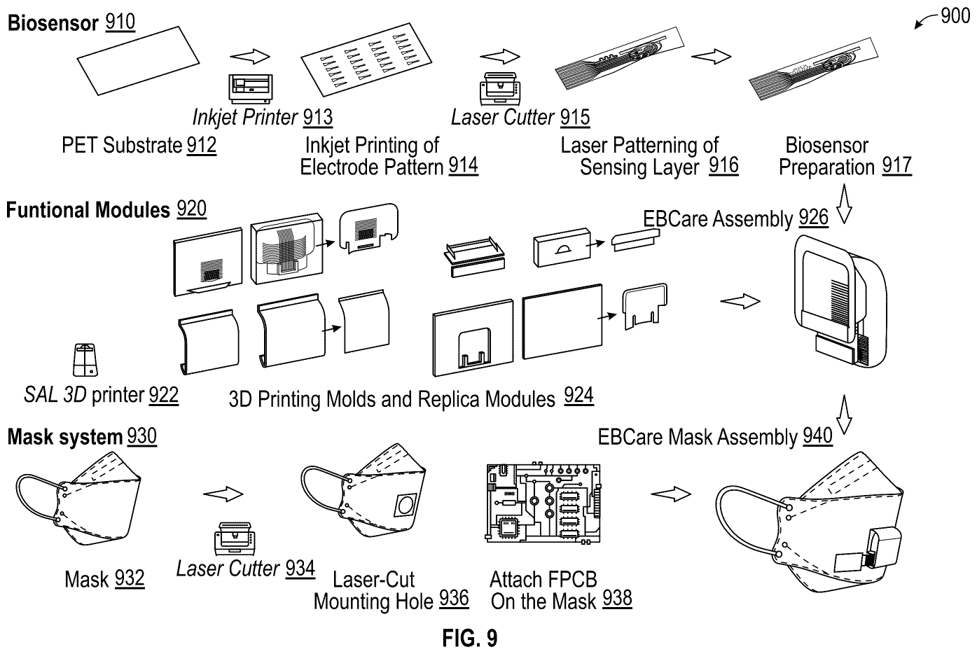

FIG. 9 is an illustration showing an example fabrication method 900 for the wearable respiratory analysis system, in accordance with various embodiments of the disclosed technology.

The fabrication method 900 may be a mold-based method that may be employed to prepare the cooling layers, microfluidic layers, and sunshield layer of the EBCare device. Firstly, three-dimensional (3D) models may be designed. Subsequently, molds may be printed by a Stereolithography (SLA) 3D printer. The obtained molds may be cleaned with isopropyl alcohol (IPA) and then exposed to a UV lamp for a period of time, followed by baking in an oven for a second period of time to allow curing of the hybrid polymers within the molds. For parts with fine micropillar structures, the reverse molding process may be repeated twice (in the first step, PDMS molds with reverse holes may be obtained). The PDMS (with, for example, a 10:1 mass ratio of prepolymer to crosslinker), PDMS-b-PEG copolymer, and Al₂O₃ microspheres may be mixed in a, for example, 50:1:50 mass ratio to create a liquid hybrid polymeric metamaterial (PDMS: PDMS-b-PEG/Al₂O₃), which may be poured into molds, whose surfaces may be sprayed with a release agent. Vacuum then may be applied to extract the gas inside. These parts may then be cured in an oven before being demolded.

The process for preparing the cooling hydrogel may be as follows: initially, agarose and Ag nanoparticles may be added to deionized (DI) water. The mixture may then be stirred while heating until it becomes homogeneous. To improve uniform dispersion, the solution may undergo an ultrasonic bath. Subsequently, the solution may be poured into 3D-printed molds and go through room temperature gelation. Finally, the hydrogel may be taken out and stored in DI water.

The fabrication method for the biosensor 910 may be depicted with relation to an inkjet printer 913 and laser cutter 915. Initially, the polyethylene terephthalate (PET) substrate 912 may be washed with IPA and then dried with compressed air flow. The multimodal biosensor 910 array may be fabricated by inkjet printing of electrode patterns 914 via a serial printing of gold (reference, counter electrodes, pH, and alcohol sensors), carbon (NH₄⁺, NO₂⁻ and temperature sensors) and SU-8 (encapsulation) layers using an inkjet printer 913. Subsequently, the printed electrodes array may be placed in an oven for a period for sintering of the biosensors pattern. Finally, the printed electrodes array bay may laser patterned 916 to separate sensors and efflux holes using a laser cutter 916.

Each individual biosensor may utilize a separate biosensor preparation 917 before EBCare assembly 926. For example, the Ag/AgCl reference electrode may be fabricated by electrodeposition of Ag on the Au electrode in a solution containing silver nitrate, sodium thiosulfate and sodium bisulfite. Electrodeposition may then be carried out using a multi-current step protocol followed by drop-casting an aliquot of iron chloride for chlorination. To prepare the reference electrode membrane, a reference solution may be prepared by dissolving PVB and fine NaCl particles into methanol. Subsequently, a PVB reference cocktail with fine NaCl particles inside may be dropcasted on the Ag/AgCl surface, followed by drop-casting of PDMS as an encapsulation. In the end, the electrode may be left to cure.

In a further example, the pH sensing electrode may be prepared by electrodeposition of polyaniline (PANI) pH sensing membrane on the inkjet-printed Au electrode by cyclic voltammetry. Whereas, in the preparation of the NH₄⁺ sensor, the NH₄⁺ selective cocktail solution may be prepared by dissolving Ammonium ionophore I, PVC, SEBS, and DOS in a THF by sonication bath. Subsequently, the NH₄⁺ selective cocktail may be drop-cast onto a 1 carbon electrode to achieve coverage. The coated electrode may then be left to dry. Additionally, for alcohol sensor fabrication, a transducer layer of Pt nanoparticles (PtNPs) on the Au electrode may firstly be electrodeposited by applying a constant voltage in an aqueous solution containing H₂PtCl₆ and formic acid. Subsequently, an Alcohol Oxidase (AOx) cocktail may be prepared as follows: chitosan may be dissolved in acetic acid, and Bovine Serum Albumin (BSA) may be dissolved in Phosphate-Buffered Saline (PBS). The chitosan and BSA solution may be mixed thoroughly with AOx. Thereafter, the AOx cocktail may be drop-casted onto the electrode surface and dried to form an enzymatic layer. Finally, an encapsulation membrane may be further drop-casted by applying polymeric solution, which may be a THF solution containing DMF and PU. Other biosensors may be fabricated using other suitable fabrication methods.

For the functional modules 920, an SAL 3D printer 922 may be used to print molds and replica modules 924, which may integrated with the biosensor to assemble the EBCare device 926. A three-dimensional modeling software may be utilized to design the three-dimensional structure of a mold 924, which may be subsequently fabricated using a 3D printer 922. The mold may undergoes ultraviolet (UV) irradiation and heating, enabling stable crosslinking of the alumina-PDMS polymer within the mold during the molding process. A liquid hybrid polymeric metamaterial may be prepared by mixing polydimethylsiloxane (PDMS) having a, for example, 10:1 mass ratio of prepolymer to crosslinker, PDMS-b-PEG copolymer, and Al₂O₃ microspheres in a, for example, 50:1:50 mass ratio, wherein the Al₂O₃ mass ratio may be optionally decreased to facilitate curing. Prior to introducing the liquid hybrid polymeric metamaterial, the surfaces of the fabricated mold may be pre-treated with a release agent to facilitate subsequent removal of cured samples. The liquid hybrid polymeric metamaterial may then be poured into the pre-treated mold, followed by application of vacuum to extract entrapped gas. Subsequently, the mold containing the hybrid polymer is heated in an oven to cure and form different polymer modules 920, after which the cured structure may be demolded.

The biosensor layer, the cooling layer, the microfluidic layer, and the sunshield layer were integrated together using silicone adhesive. Subsequently, cooling hydrogel may be installed to complete the assembly of the EBCare device.

For the mask body 932, a laser cutter 934 may be used to cut a mounting hole 936 into the mask. A flexible printed circuit board 938 (FPCB) and battery may then be installed within the interlayers of the mask. Using medical tape cut with a laser cutter, for EBCare/wearable respiratory analysis system assembly 940, the edges of EBCare device may be sealed to the mounting hole in the mask, followed by connecting the FPCB with the biosensor interface.

FIG. 10 is an illustration showing an example FPCB, in accordance with various embodiments of the disclosed technology. FIG. 10 depicts an example FPCB 1002, an example wireless application 1004-1009, and a logic circuit 1010-1040.

To realize wireless multiplexed wearable EBC analysis, a FPCB 1002 may be used for multimodal electrochemical measurements (e.g., voltammetry, potentiometry, and impedimetry), signal processing, and wireless communication. Real-time or near-real-time collected analyte information can be transmitted to a user interface 1040 through Bluetooth Low Energy (BLE) 1016 and displayed on a custom-developed mobile app 1004-1009. As the wearable respiratory analysis system uses a multiplexed circuit for continuous monitoring, the sampling rate can be programmatically adjusted to any desired frequency below 100 Hz. The wearable respiratory analysis system may include various operational modes to optimize power consumption, including: running mode, low-power mode, and/or lower-power intermittent mode. These modes can adjust the electrochemical measurement and data collection processes, enabling efficient, long-term continuous sensing. The fully integrated wearable respiratory analysis system can accurately and simultaneously monitor dynamic responses of the integrated NO₂⁻, alcohol, NH₄⁺, pH, and temperature sensors (e.g., sensors 504-514 of FIG. 5); these biosensors may provide for stability during continuous microfluidic sensing and selectivity to other interferent molecules.

The wireless application may include a customized mobile app 1004, which may display various analyte concentrations or metrics 1005-1009 within the app. The displayed analyte concentrations and/or metrics 1005-1009 may include concentrations or metrics representative of any biomarker discussed herein or any biomarker that may be added to the wearable respiratory analysis system.

Additionally, the logic circuit may include a processor and a non-transitory memory with computer executable instructions embedded thereon. The computer executable instructions may instruct the electrode in the biosensor to take a measurement of an electrical property of the recognition layer. This measurement may be representative of concentrations or presence of target biomarkers. The logic circuit may further be electrically coupled to the electrode and the computer executable instructions may cause the processor to identify the electrical property detected with the electrode when the target biomarkers interact with the recognition layer.

The logic circuit can be broadly categorized into three functional parts: power and sensor ports, data processing and wireless communication, and electrochemical instrumentation. To power the circuit, a power supply 1010 may supply voltage, which may be regulated through a voltage regulator. Data processing and wireless communication may be executed by a compact wireless module featuring an integrated Microcontroller Unit 1012 (MCU) and Bluetooth Low Energy 1016 (BLE) radio. The reference potential of the reference electrode 1036 may be upheld by a constant voltage chip 1018. For open-circuit potential (OCPT) measurements, the voltage between the working 1032, reference 1036, and control electrodes 1038 may be amplified through Instrumentation Amplifiers 1024 (INA) and then read by the MCU's 1012 Analog-to-Digital Converter 1014 (ADC) peripheral. In amperometric scans, the MCU 1012 may control the digital-to-analog 1020 (DAC) converters through the Serial Peripheral Interface (SPI) protocol or potentiostat 1028, outputting a constant working potential for the working electrodes 1034. The current through the working electrodes 1034 may be amplified by the trans-impedance amplifier 1022 (TIA) module and converted to a voltage, which may then read by the MCU's 1012 ADC 1014 peripheral. The MCU 1012 could also measure the resistance of the temperature sensor 1030 using a voltage divider 1026 circuit and the built-in ADC 1014. The acquired multimodal data was wirelessly transmitted via BLE 1016 to the user's mobile device/user interface 1040 and further calibrated and analyzed by custom-developed software.

The wearable respiratory analysis system may employ various power sources. For example, in one embodiment, the system may be equipped with a lightweight battery. In another embodiment, the system may be wired to an external device's power supply. In another embodiment, the system may leverage a biofluid powering system to power the device with the collected EBC flow itself. In another embodiment, the system may be powered with a small solar panel. In another embodiment, the wearable assessment system may be powered by human motion.

While the wearable respiratory system is discussed in relation to the aforementioned biomarkers, the system may further include various other biomarker measurements. For example, the system may measure the concentrations of all or any of the nine essential amino acids. Amino acids are organic compounds that are present in the human body and in food sources. Concentrations of amino acids may vary depending on many factors including dietary intake, genetic predisposition, gut microbiota, environmental factors, lifestyle factors including sleep and exercise, and other factors. The concentrations of amino acids present in human bodily fluids, including EBC, can provide important information about the health of an individual. For example, elevated levels of branched-chain amino acids (BCAAs) including for example, leucine (Leu), isoleucine (Ile), and valine (Val) may be correlated with certain health conditions including obesity, insulin resistance, diabetes, cardiovascular disease, and pancreatic cancer. Deficiencies in amino acids, including, for example, arginine and cysteine, may indicate immune suppression and/or reduced immune-cell activation

In another embodiment, the system may measure acetone in EBC. Acetone may be a promising biomarker for various physiological and pathological conditions, offering potential for non-invasive diagnostic applications. The presence and concentration of acetone in EBC may correlate with several metabolic processes, particularly ketone body production and fatty acid metabolism. Research has indicated that elevated acetone levels in EBC may serve as an indicator for conditions such as uncontrolled diabetes mellitus, where impaired glucose metabolism may lead to increased ketogenesis.

In another embodiment, the system may measure concentrations of amino acids in addition to other organic compounds, including vitamins and minerals. For example, imbalances with tryptophan (Trp), tyrosine (Tyr) and phenylalanine (Phe), which are needed to support neurotransmitters such as serotonin, dopamine, norepinephrine, and epinephrine, may indicate neurological and/or mental health conditions. Other metabolic indicators involving, for example, Leu, Phe, and vitamin D, may be linked with severity, vulnerability, and mortality related to viral infections including COVID-19. Other compounds, like glucose and uric acid may also be measured to determine risk of developing, and/or severity of, a particular health condition.

In another embodiment, amino acids, vitamins, and mineral concentrations may be measured to develop a personalized nutrition plan. After measurement of initial concentrations, a human patient may be advised to make dietary modifications to account for deficiencies and/or excesses of key amino acids, vitamins, and minerals. The human patients adherence to a nutritional plan and progress may be monitored continuously with the system.

As discussed throughout, health status detection and evaluation may be made based on concentrations of relevant biomarkers. An object model for health status may be trained. For example, the object model may be trained with standard health status questionnaires. Then, machine learning methods may be used to optimize detection and evaluation of health status through biomarker analysis, using questionnaires as an object model. For example, a machine learning model may optimize which biomarkers are most accurately correlated with health status determinations. A machine learning model may further optimize the level of detected biomarkers that correlate more accurately to noteworthy health conditions.

In another embodiment a wearable system may detect and measure drug/alcohol compounds present in a EBC. Drug/alcohol compounds may be measured to assess compliance with a drug/alcohol treatment regimen. Drug compounds may also be measured to assess successful metabolization of a treatment drug. Drug compounds may also be measured to determine the risk and/or severity of drug toxicity due to a drug treatment regimen.

In another embodiment, the system may measure the concentration of certain hormones. In another embodiment, the system may measure the concentration of antibodies present in a human patient which may indicate an infection, the degree of immune response to a viral, bacterial, or fungal agent, an autoimmune disease, or another health condition.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A wearable respiratory analysis system, comprising:
a mask body, a fluid transport system, a biosensor, a logic circuit, and a transceiver;
wherein the mask body is shaped to cover a portion of a user's face and capture respiratory emissions from a user, and comprises a cooling layer to condense the respiratory emissions into liquid droplets;
the fluid transport system coupling the mask body to the biosensor, wherein the biosensor comprises a recognition layer comprising a bioreceptor and an electrode electrically coupled to the recognition layer; and
the biosensor generating a signal indicating a concentration of a biomarker within the liquid droplets and passing the signal to the logic circuit, and the logic circuit causing the transceiver to transmit the signal to a remote device.

2. The wearable respiratory analysis system of claim 1, wherein the cooling layer is passive and comprises at least one of evaporative cooling, radiative cooling, thermoelectrical cooling, and cooling via a hydrophilic surface material.

3. The wearable respiratory analysis system of claim 1, wherein the fluid transport system comprises capillaries ranging in diameter from 1 micrometers to 1000 micrometers, which are shaped to cause a capillary transport effect to transport the liquid droplets from the mask body to the biosensor.

4. The wearable respiratory analysis system of claim 1, wherein the biosensor comprises an electrochemical sensor to detect a target biomarker, wherein the target biomarker is selected from a group consisting of nitrites, ammonia, cytokines, acetone, lactate, tuberculosis biomarkers, hydrogen peroxide, nitrates, alcohol, volatile organic compounds, lipids, proteins, DNA, RNA, fatty acids, and viral pathogens.

5. The wearable respiratory analysis system of claim 1, wherein the mask body further comprises a sunshield comprising a hybrid metamaterial selected from the group consisting of metallic materials, silver, aluminum, oxide materials, titanium dioxide, zirconium dioxide, polymer materials, ceramic polymer-hybrid, aluminum oxide, polyvinylidene fluoride (PVDF), polyethylene (PE), polydimethylsiloxane (PDMS), and copolymer PDMS-block-polyethylene glycol (PDMS-b-PEG).

6. The wearable respiratory analysis system of claim 1, wherein the cooling layer comprises a first sub-layer, a second sub-layer, and a polydimethylsiloxane (PDMS) matrix,
wherein the first sub-layer is evaporative and comprises a hydrogel and the second sub-layer is radiative and comprises aluminum oxide.

7. The wearable respiratory analysis system of claim 1, further comprising a logic circuit comprising a processor and a non-transitory memory with computer executable instructions embedded thereon;
wherein the electrode is configured to detect a measurement of an electrical property of the recognition layer; and
wherein the logic circuit is electrically coupled to the electrode and the computer executable instructions cause the processor to identify the electrical property detected with the electrode when the target biomarkers interacts with the recognition layer.

8. A wearable smart mask analysis system, comprising:
a mask body having a geometry to cover a user's mouth and nose, the mask body comprising a breath condensation layer to capture exhaled breath condensate (EBC);
a tandem cooling layer embedded within the mask body, the tandem cooling layer comprising a hydrogel evaporative cooling sub-layer, a metamaterial radiative cooling sub-layer, and a thermal conductive framework;
a microfluidic capillary system within the mask body to capture and transport EBC, the microfluidic capillary system comprising graded capillary channels further comprising a microengineered pillar array and hydrophilic microfluidic channels;
a nanoengineered electrochemical biosensor array coupled to the microfluidic capillary system for selective and sensitive analysis of biomarkers present in the EBC; and
a flexible printed circuit board (FPCB) interfaced with the electrochemical biosensor array for signal processing and wireless communication.

9. The system of claim 8, wherein the breath condensation layer comprises a hydrophilic surface.

10. The system of claim 8, wherein the hydrogel evaporative cooling sub-layer comprises agarose hydrogel doped with silver nanoparticles.

11. The system of claim 8, wherein the microfluidic capillary system further comprises an evaporative cooling hydrogel top-layer covering microchannels on an outer surface of the mask body.

12. The system of claim 8, wherein the nanoengineered electrochemical biosensor array comprises biosensors to detect a target biomarker, wherein the target biomarker is selected from a group consisting of nitrites, ammonia, cytokines, acetone, lactate, tuberculosis biomarkers, hydrogen peroxide, nitrates, alcohol, volatile organic compounds, lipids, proteins, DNA, RNA, fatty acids, and viral pathogens.

13. The system of claim 8, further comprising
a recognition layer comprising a bioreceptor selected from the group consisting of: an enzyme, an ion-selective molecule, an imprinted polymer, and an antibody, wherein the bioreceptor selectively interacts with a target biomarker in EBC;
an electrode configured to detect a measurement of an electrical property of the recognition layer;
a logic circuit comprising a processor and a non-transitory memory with computer executable instructions embedded thereon; and
wherein the logic circuit is electrically coupled to the electrode and the computer executable instructions cause the processor to identify the electrical property detected with the electrode when the target biomarker interacts with the bioreceptor.

14. The system of claim 8, further comprising a sunshield layer incorporated into the mask body, wherein the sunshield layer comprises a ceramic alumina-polymer hybrid metamaterial.

15. A method for exhaled breath condensate (EBC) analysis, comprising:
providing a wearable mask having a geometry to cover a user's respiratory outlets, wherein the wearable mask comprises:
a mask body comprising a passive cooling system for condensing exhaled breath into EBC;
a microfluidics system for directing the EBC to a designated analysis area within the mask body;
an integrated biosensor array for detecting biomarkers within the EBC; and
a communication module for transmitting analysis results;
capturing exhaled breath by the wearable mask and condensing it into EBC using the passive cooling system, wherein the passive cooling system comprises a tandem cooling layer further comprising an evaporative cooling hydrogel sub-layer, radiative cooling sub-layer, and a hydrophilic surface materials;
transporting the condensed EBC through the microfluidic system to the integrated biosensor array by leveraging fluid capillary forces induced by graded pillar structures within the wearable mask;
analyzing the EBC in situ with the integrated biosensor array to detect and quantify biomarkers indicative of a user's respiratory health;
transmitting the results of analyzing the EBC from the wearable mask to an external receiver via the communication module; and
refreshing the hydrogel sub-layer of the passive cooling system through microfluidic transport of EBC.

16. The method of claim 15, wherein the passive cooling system's evaporative cooling hydrogel sub-layer comprises a hydrogel infused with antimicrobial agents.

17. The method of claim 15, wherein analyzing the EBC includes employing electrochemical sensors within the biosensor array to perform a multiplexed analysis.

18. The method of claim 15, further comprising calibrating the biosensor array before analyzing the EBC, wherein calibrating the biosensor array comprises adjusting biosensor responses based on known concentrations of analytes.

19. The method of claim 15, wherein transporting the condensed EBC through the microfluidic system to the integrated biosensor array comprises using a microengineered gradient in pillar height and density within the microfluidic system.

20. The method of claim 15, wherein transmitting the results of analyzing the EBC comprises transmitting the results using a low-energy wireless protocol to send data to an external device.
